(19) United States
(12) Patent Application Publication
(10) Pub. No.: US 2025/0009303 A1
(43) Pub. Date: Jan. 9, 2025

(54) INFANT AND CHILD MONITORING SYSTEM, INTELLIGENT CHANGING PAD SYSTEM, HEALTH TRACKING SYSTEM, AND METHODS OF USE

(71) Applicant: Shaker Rawanbakhsh, Alamo, CA (US)

(72) Inventor: Shaker Rawanbakhsh, Alamo, CA (US)

(21) Appl. No.: 18/595,393

(22) Filed: Mar. 4, 2024

Related U.S. Application Data
(60) Provisional application No. 63/488,474, filed on Mar. 3, 2023.

(51) Int. Cl. A61B 5/00; A61B 5/01; G01G 19/44; G01G 19/50

(52) U.S. Cl. CPC A61B 5/6892; A61B 5/002; A61B 5/0022; A61B 5/01; A61B 5/4875; A61B 5/7405; A61B 5/742; A61B 2503/045; A61B 2562/029; G01G 19/445; G01G 19/50

(57) ABSTRACT

Methods, systems and apparatuses for infant and child monitoring, for changing pads, and for tracking heath. One system is an intelligent changing pad system for infants and children that includes a main body with a changing surface, a scale integrated within the changing surface for measuring weight, a plurality of sensors configured to monitor health-related metrics of a human including temperature and hydration levels, a graphical user interface for displaying the human's health metrics and alerts, and a wireless communication system for transmitting the monitored data to a remote computing device.

### CROSS-REFERENCES TO RELATED PATENT APPLICATIONS

This application claims the benefit of U.S. Provisional Application No. 63/488,474, filed on Mar. 3, 2023, the entire contents of which is incorporated herein by reference.

### STATEMENT REGARDING FEDERALLY SPONSORED RESEARCH OR DEVELOPMENT

### Not Applicable.

### REFERENCE TO SEQUENCE LISTING, A TABLE, OR A COMPUTER PROGRAM LISTING COMPACT DISK APPENDIX

Not Applicable.

### BACKGROUND

Embodiments of the inventive subject matter relate to relate to infant and child monitoring systems, intelligent changing pad systems, health tracking systems, and methods of use. Embodiments also relate to an infant monitoring system which monitors and records various metrics and/or information related to an infant. Embodiments also relate to a child monitoring system which monitors and records various metrics and/or information related to a child. Embodiments also relate to an intelligent changing pad system. Embodiments also relate to an infant and/or child health tracking system. However, the present disclosure is not limited to these novel and inventive apparatuses, systems and improvements and methods of use, and the disclosed embodiments may further be varied and adapted for a variety of purposes.

Infants and children have always required care from adults and/or other caregivers. The needs of an infant are many and include, but are not limited to, warmth, rest or sleep, food and nutrition, hygiene, and more. Caring for an infant and/or child is not an easy task. Caring for an infant and/or child takes constant attention, constant and/or ongoing precautions, and the engagement of various activities by adults and/or caregivers.

For this reason, infant and/or child caregivers often seek out training, educational tools, other tools, services, and/or products and methods which make caring for an infant and/or child easier. In addition to improving upon the ease by which an adult and/or caregiver can provide care, various tools, services and/or products may also be beneficial for the health and/or wellbeing of the infant and/or child.

The prior art includes a wide variety of tools and/or products which make infant and/or child caring easier. A few examples of some of these tools and/or products includes baby ribs, baby bottles, food utensils (and specialized food for various ages), strollers, changing tables, changing pads, and the like. These tools and/or products, among other tools and products, have improved infant and/or childcare tremendously over the years.

Infant hygiene is an especially important part of infant and/or toddler care. Reusable cloth diapers and/or disposable diapers form a common means of maintaining one aspect of infant hygiene. Regardless of choice, both types of diapers need to be changed regularly. A sanitary surface is needed to place an infant on for the duration of the changing process. Many problems plague the art in the goal of achieving a sanitary and comfortable environment for an infant during what can be an uncomfortable process.

Caregivers commonly provide diaper hanging and other services on beds, or in cribs, or at best on a changing table. A changing table, commonly known in the art, is a raised platform designed to aid a caregiver in changing a diaper or clothes of an infant. Changing tables, known in the art, are raised to bring the infant to an easy to access height for a user. In this way a user can easily access and/or reach the infant. Changing tables are commonly made of hard plastic and are also known in public places as being available in public restrooms.

Commonly, changing tables are formed of a plastic platform which may even fold down on hinges so it can be stowed when not in use. Changing pads are another tool designed to aid a parent and/or caregiver in maintaining infant hygiene. Changing pads are softer surfaces which provide a barrier between an infant and the surface the infant is resting on. In this way, changing pads, as known in the art, provide a barrier between a bed, crib, and the like to both protect the infant from contaminants of the surface as well as to protect the surface from contaminants from the infant. Similarly, changing pads can provide a light layer of cushion between an infant and the hard plastic surfaces often available and/or used for hygiene purposes.

While the prior art includes a number of changing pads or changing sheets, the current art is limited with capability and faces a number of long felt needs. Known changing pads and/or changing sheets are uncomfortable for infants and/or children, they lack sanitary assurances needed by caregivers, and more. Similarly, changing tables are limited in comfort and functionality.

Thus, the present disclosure provides embodiments for an infant and child monitoring system, an intelligent changing pad system, a health tracking system, and methods of use that improve upon the state of the art and introduce novel functionality, components, and systems.

### SUMMARY

The illustrative embodiments provide embodiments including an infant and child monitoring system, an intelligent changing pad system, a health tracking system, and methods of use that improve upon the prior art and introduce novel functionality, components, and systems. In one aspect, the present disclosure provides embodiments for a new and useful infant and child monitoring system. In another aspect, the present disclosure provides a new and useful intelligent changing pad system. The present disclosure provides embodiments for a new and useful health tracking system. Furthermore, the present disclosure provides new and useful methods of use.

The intelligent changing pad system may be implemented when changing newborns, infants and/or children, as well as for other purposes. The present disclosure also provides for monitoring and tracking of infant and/or child health. In this way, the present disclosure provides for monitoring and tracking, especially important data, that a caregiver, medical provider and the like may need to assist in providing care for an infant and/or newborn and/or child. Additionally, the present disclosure provides embodiments including a safe intelligent changing pad system. Furthermore, in this way, the present disclosure provides the state of the art with a comfortable space for an infant and/or child. Furthermore, the present disclosure provides the state of the art with a comfortable space for an infant and/or child that is safe and comfortable.

Said another way, the present disclosure provides the state of the art with a smart change pad that provides for tracking and/or monitoring infant and/or child growth. This is especially critical during the early period of human lives. Similarly, the present disclosure tracks and monitors this information for parents and/or caregivers and may also provide alerts and/or comparisons to normal data. Additionally, and similarly, the present disclosure provides a means for manual data entry so that various data can be tracked, analyzed, compared, and the like so that information can be processed and provided.

In one embodiment, the present disclosure provides a high accuracy scale, which is critical in the data tracking of newborns or infants. In this way, health and even small variations in the weight of an infant could be critical. Similarly, the high accuracy scale also helps track food intake and development of an infant and/or child, and the like.

Similarly, the present disclosure also provides for graphical user interfaces, mobile phone applications, personal computer interfaces, tablet applications, and the like which provide for recording various information, storing this data, and providing this data to a user and/or healthcare provider, and/or subsequent and other caregivers. In addition to tracking measurements, weights, and the like, the present disclosure also provides event tracking and other similar data tracking, including but not limited to feeding occurrences, diaper change occurrences, as well as providing illustrations, videos and such to aid a user in the task at hand or in other learning tasks. In this way, the present disclosure can overlay data such as occurrences and weight and provide further analysis and information to a user. Similarly, the system also provides knowledge and/or education to a user throughout a process if desired. Similarly, graphics and other helpful information such as imposed overlayment of a standard or a standard deviation in a graph and other display information is provided herein.

Furthermore, the present disclosure provides for additional components, features, and functionality including built-in interface and touchscreens providing for user input and other capabilities. Furthermore, the present disclosure provides for additional components, features and functionality directed toward visibility. These features include, but are not limited to, a night light and other viewing features that can aid a user in various environments, further easing stress, anxiety, and operation. Furthermore, the present disclosure provides for additional components, features and functionality directed toward the noise environment. These features include, but are not limited to, a white noise machine and components and other features which aid in creating a soothing environment. Furthermore, the present disclosure provides for additional components, features and functionality directed toward temperature stimulus. These features include, but are not limited to, warming components, sensors, and features. Furthermore, the present disclosure provides for additional components, features and functionality directed toward sanitary hygiene. In this way, some features include, but are not limited to a removable and washable cover and/or pad device that is easy to clean and improves upon overall health. Furthermore, the present disclosure provides for additional components, features and functionality directed toward security and stability. In this way, some features include, but are not limited to securement features and the like.

Furthermore, the present disclosure provides embodiments with support including but not limited to, recommendations, tips, insights, blogs, videos, local and national data, trends, patterns, video conferencing with physicians and others, among various other components, functionality, and features.

Thus, it is a primary object of the inventive subject matter to provide an infant and child monitoring system, an intelligent changing pad system, a health tracking system, and methods of use that provide a novel system for the state of the art in addition to improving upon the prior art.

Another object of the disclosure is to provide an infant and child monitoring system, an intelligent changing pad system, a health tracking system, and methods of use that provide a smart change pad.

Yet another object of the disclosure is to provide an infant and child monitoring system, an intelligent changing pad system, a health tracking system, and methods of use that provide educational tools and features.

Another object of the disclosure is to provide an infant and child monitoring system, an intelligent changing pad system, a health tracking system, and methods of use that provide communication features and functionality.

Yet another object of the disclosure is to provide an infant and child monitoring system, an intelligent changing pad system, a health tracking system, and methods of use that provide infant and/or child monitoring.

Another object of the disclosure is to provide an infant and child monitoring system, an intelligent changing pad system, a health tracking system, and methods of use that provide infant and/or child data tracking.

Yet another object of the disclosure is to provide an infant and child monitoring system, an intelligent changing pad system, a health tracking system, and methods of use that provide infant and/or child growth tracking.

Another object of the disclosure is to provide an infant and child monitoring system, an intelligent changing pad system, a health tracking system, and methods of use that provide infant and/or child weight tracking.

Yet another object of the disclosure is to provide an infant and child monitoring system, an intelligent changing pad system, a health tracking system, and methods of use that provide infant and/or child dimension tracking.

Another object of the disclosure is to provide an infant and child monitoring system, an intelligent changing pad system, a health tracking system, and methods of use that provide event occurrence tracking.

Yet another object of the disclosure is to provide an infant and child monitoring system, an intelligent changing pad system, a health tracking system, and methods of use that provide a high accuracy scale.

Another object of the disclosure is to provide an infant and child monitoring system, an intelligent changing pad system, a health tracking system, and methods of use that provide accompanying applications for use on a personal computer device, smart phone, tablet, or the like.

Yet another object of the disclosure is to provide an infant and child monitoring system, an intelligent changing pad system, a health tracking system, and methods of use that aids a caregiver in changing clothes of an infant.

Another object of the disclosure is to provide an infant and child monitoring system, an intelligent changing pad system, a health tracking system, and methods of use that provide data management interface, memory, and processing capabilities.

Yet another object of the disclosure is to provide an infant and child monitoring system, an intelligent changing pad system, a health tracking system, and methods of use that provide support blogs.

Another object of the disclosure is to provide an infant and child monitoring system, an intelligent changing pad system, a health tracking system, and methods of use that provide insights.

Yet another object of the disclosure is to provide an infant and child monitoring system, an intelligent changing pad system, a health tracking system, and methods of use that provide tips.

Another object of the disclosure is to provide an infant and child monitoring system, an intelligent changing pad system, a health tracking system, and methods of use that provide recommendations.

Yet another object of the disclosure is to provide an infant and child monitoring system, an intelligent changing pad system, a health tracking system, and methods of use that provide access to local data.

Another object of the disclosure is to provide an infant and child monitoring system, an intelligent changing pad system, a health tracking system, and methods of use that provide access to national data.

Yet another object of the disclosure is to provide an infant and child monitoring system, an intelligent changing pad system, a health tracking system, and methods of use that provide trend data.

Another object of the disclosure is to provide an infant and child monitoring system, an intelligent changing pad system, a health tracking system, and methods of use that provide patterns.

Yet another object of the disclosure is to provide an infant and child monitoring system, an intelligent changing pad system, a health tracking system, and methods of use that provide note taking capabilities.

Another object of the disclosure is to provide an infant and child monitoring system, an intelligent changing pad system, a health tracking system, and methods of use that provide visual support features.

Yet another object of the disclosure is to provide an infant and child monitoring system, an intelligent changing pad system, a health tracking system, and methods of use that allows a user to operate at night and/or in low light operating conditions.

Another object of the disclosure is to provide an infant and child monitoring system, an intelligent changing pad system, a health tracking system, and methods of use that provide temperature settings for comfort and safety improvements.

Yet another object of the disclosure is to provide an infant and child monitoring system, an intelligent changing pad system, a health tracking system, and methods of use that provide ergonomic improvements for a user.

Another object of the disclosure is to provide an infant and child monitoring system, an intelligent changing pad system, a health tracking system, and methods of use that provide ergonomic improvements for an infant and/or child.

Yet another object of the disclosure is to provide an infant and child monitoring system, an intelligent changing pad system, a health tracking system, and methods of use that provide movement capabilities such as vibration and the like.

Another object of the disclosure is to provide an infant and child monitoring system, an intelligent changing pad system, a health tracking system, and methods of use that provide audio stimulus, such as white noise maker and associated components.

Yet another object of the disclosure is to provide an infant and child monitoring system, an intelligent changing pad system, a health tracking system, and methods of use that provide for various securement devices.

Another object of the disclosure is to provide an infant and child monitoring system, an intelligent changing pad system, a health tracking system, and methods of use that provide a retractable belt for safety of the infant and other users.

Yet another object of the disclosure is to provide an infant and child monitoring system, an intelligent changing pad system, a health tracking system, and methods of use that provide bluetooth or other communication connections for sending and/or receiving data and/or communicating with various parties.

Another object of the disclosure is to provide an infant and child monitoring system, an intelligent changing pad system, a health tracking system, and methods of use that provide for digestion and/or consumption tracking.

Yet another object of the disclosure is to provide an infant and child monitoring system, an intelligent changing pad system, a health tracking system, and methods of use that provide for feeding means and methods tracking.

Another object of the disclosure is to provide an infant and child monitoring system, an intelligent changing pad system, a health tracking system, and methods of use that provide sanitary improvements upon the state of the art.

Yet another object of the disclosure is to provide an infant and child monitoring system, an intelligent changing pad system, a health tracking system, and methods of use that provide removable cushions and/or covers.

Another object of the disclosure is to provide an infant and child monitoring system, an intelligent changing pad system, a health tracking system, and methods of use that provide washable cushions and/or covers.

Yet another object of the disclosure is to provide an infant and child monitoring system, an intelligent changing pad system, a health tracking system, and methods of use that provide antimicrobial materials for cushion and/or cushion covers.

Another object of the disclosure is to provide an infant and child monitoring system, an intelligent changing pad system, a health tracking system, and methods of use that provide water resistant features.

Yet another object of the disclosure is to provide an infant and child monitoring system, an intelligent changing pad system, a health tracking system, and methods of use that provide bisphenol A free features.

Another object of the disclosure is to provide an infant and child monitoring system, an intelligent changing pad system, a health tracking system, and methods of use that provide a plurality of sensors.

Yet another object of the disclosure is to provide an infant and child monitoring system, an intelligent changing pad system, a health tracking system, and methods of use that provide heartbeat sensing and tracking.

Another object of the disclosure is to provide an infant and child monitoring system, an intelligent changing pad system, a health tracking system, and methods of use that provides connections to various features.

Yet another object of the disclosure is to provide an infant and child monitoring system, an intelligent changing pad system, a health tracking system, and methods of use that provide noise detection.

Another object of the disclosure is to provide an infant and child monitoring system, an intelligent changing pad system, a health tracking system, and methods of use that remembers optimal settings of systems to provide maximum comfort as associated with a decibel level detection and history.

Yet another object of the disclosure is to provide an infant and child monitoring system, an intelligent changing pad system, a health tracking system, and methods of use that provide a wipe warmer.

Another object of the disclosure is to provide an infant and child monitoring system, an intelligent changing pad system, a health tracking system, and methods of use that provide a diaper holder.

Yet another object of the disclosure is to provide an infant and child monitoring system, an intelligent changing pad system, a health tracking system, and methods of use that provide a dispenser system.

Another object of the disclosure is to provide an infant and child monitoring system, an intelligent changing pad system, a health tracking system, and methods of use that provide a disposal system.

Yet another object of the disclosure is to provide an infant and child monitoring system, an intelligent changing pad system, a health tracking system, and methods of use that provide a built-in touch screen.

Another object of the disclosure is to provide an infant and child monitoring system, an intelligent changing pad system, a health tracking system, and methods of use that are environmentally friendly.

Yet another object of the disclosure is to provide an infant and child monitoring system, an intelligent changing pad system, a health tracking system, and methods of use that are easy to use.

Another object of the disclosure is to provide an infant and child monitoring system, an intelligent changing pad system, a health tracking system, and methods of use that are safe to use.

Yet another object of the disclosure is to provide an infant and child monitoring system, an intelligent changing pad system, a health tracking system, and methods of use that are fun to use.

Another object of the disclosure is to provide an infant and child monitoring system, an intelligent changing pad system, a health tracking system, and methods of use that are educational.

Yet another object of the disclosure is to provide an infant and child monitoring system, an intelligent changing pad system, a health tracking system, and methods of use that improve upon the wellbeing of a caregiver.

Another object of the disclosure is to provide an infant and child monitoring system, an intelligent changing pad system, a health tracking system, and methods of use that improve upon the wellbeing of an infant and/or child.

Yet another object of the disclosure is to provide an infant and child monitoring system, an intelligent changing pad system, a health tracking system, and methods of use that improve upon the knowledge available to a user about an infant and/or child.

Another object of the disclosure is to provide an infant and child monitoring system, an intelligent changing pad system, a health tracking system, and methods of use that provide a dramatic change in improvements in infant care.

Systems that embody the disclosure, in accordance with the aspects thereof, are typically designed by describing their functions in hardware description languages. Therefore, the present disclosure is also embodied in such hardware descriptions, and methods of describing systems as such hardware descriptions, but the scope of the present disclosure is not limited thereby. Therefore, these and other objects of the disclosure are hereby considered, not only those limited to those found in this summary of disclosure section, but as can be found throughout the scope of this specification and accompanying documents.

### DETAILED DESCRIPTION OF THE DRAWINGS

The drawings accompanying and forming part of this specification are included to depict certain aspects of the disclosure wherein:

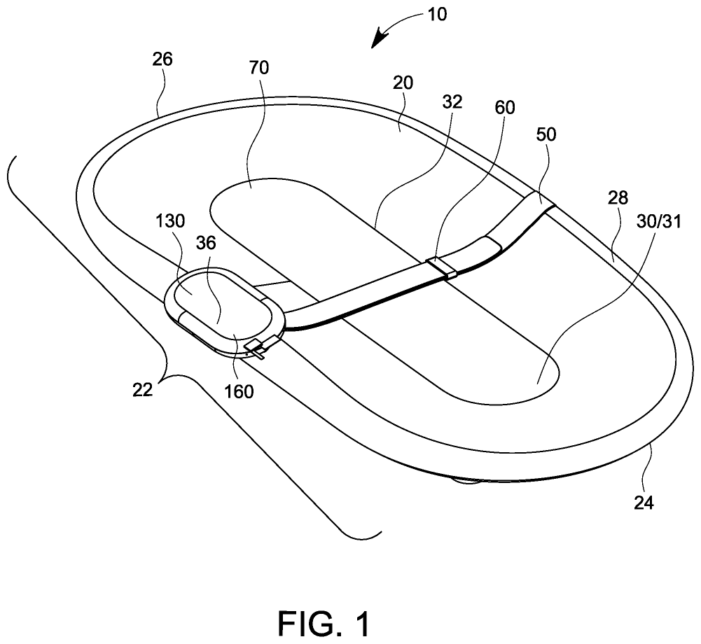

FIG. 1 shows an elevated front side view of an exemplary apparatus according to embodiments of the inventive subject matter;

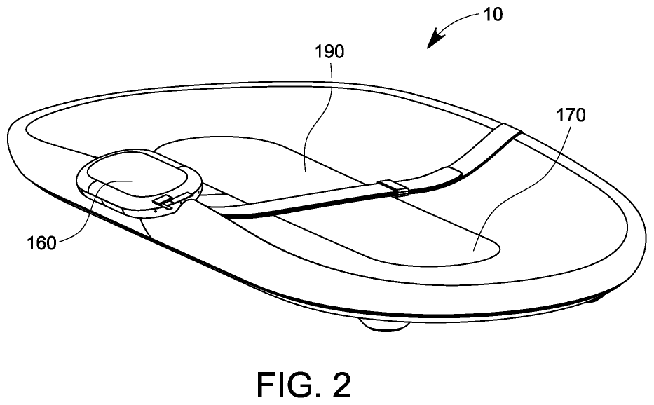

FIG. 2 shows another elevated front side view of an apparatus according to embodiments of the inventive subject matter;

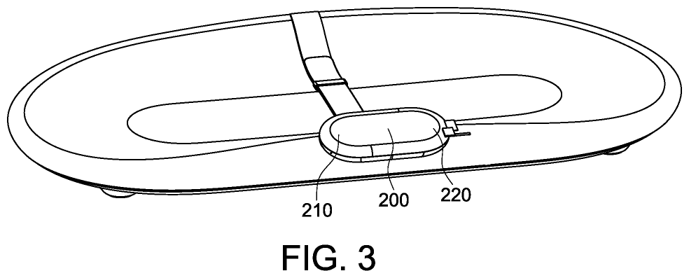

FIG. 3 illustrates another elevated front side view of an apparatus according to embodiments of the inventive subject matter;

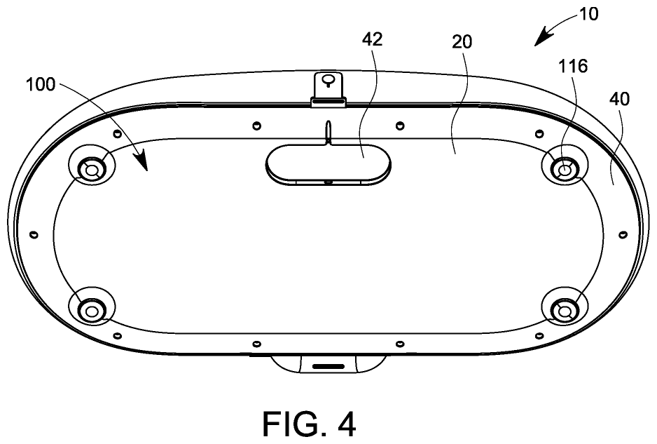

FIG. 4 shows an elevated underside view of an apparatus according to embodiments of the inventive subject matter;

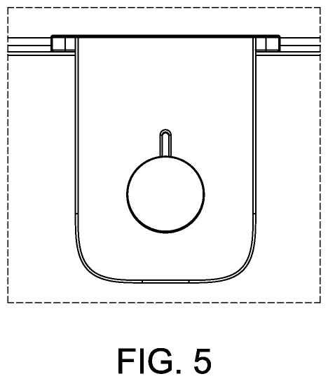

FIG. 5 shows a close up view of the side of an apparatus according to embodiments of the inventive subject matter;

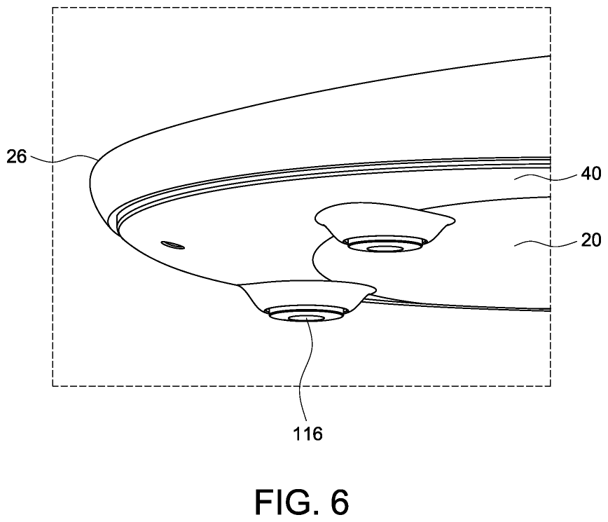

FIG. 6 shows an partial side view of an apparatus according to embodiments of the inventive subject matter;

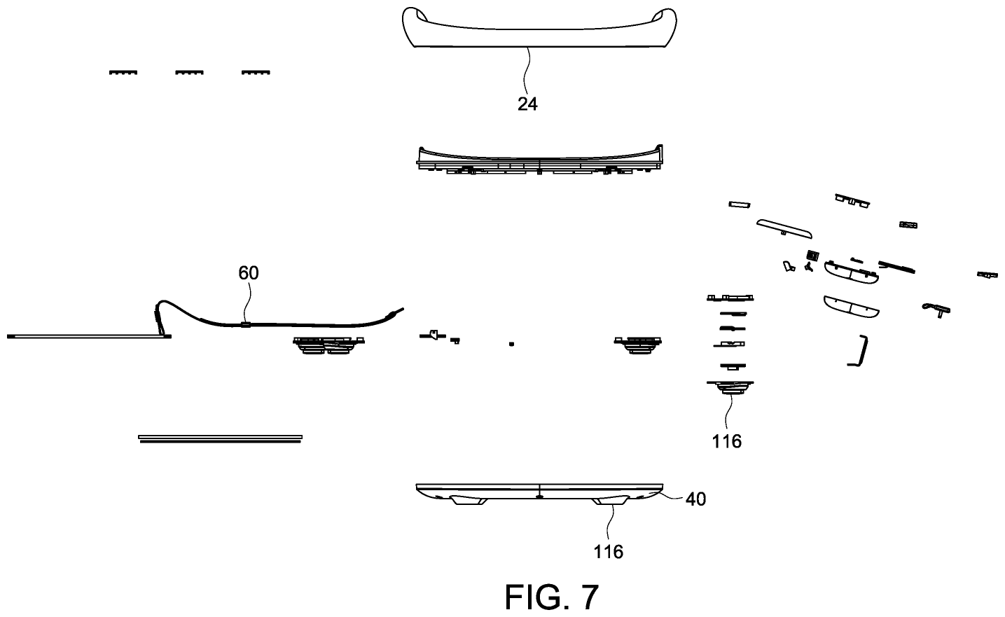

FIG. 7 shows an exemplary breakdown of parts used for an apparatus according to embodiments of the inventive subject matter;

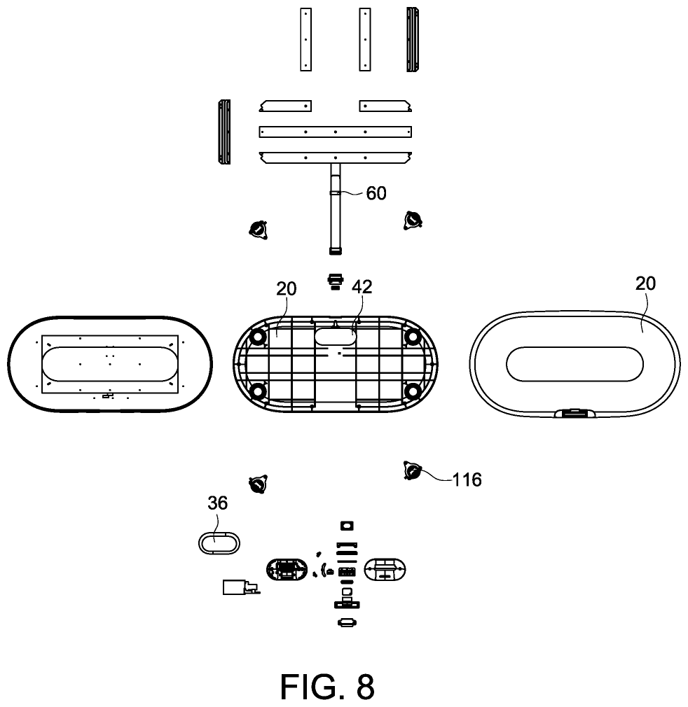

FIG. 8 shows another exemplary breakdown of parts used for an apparatus according to embodiments of the inventive subject matter;

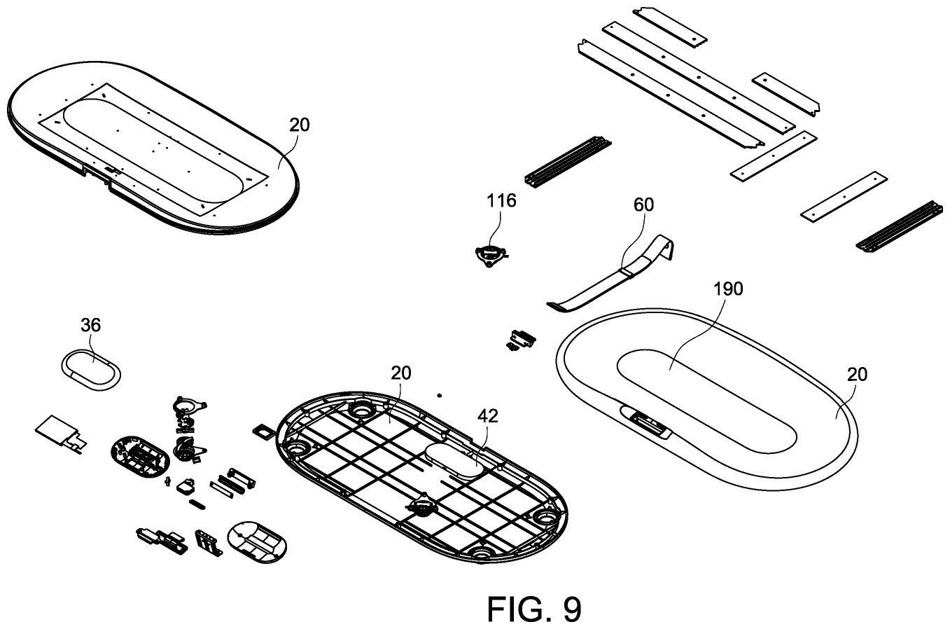

FIG. 9 shows another exemplary breakdown of parts used for an apparatus according to embodiments of the inventive subject matter;

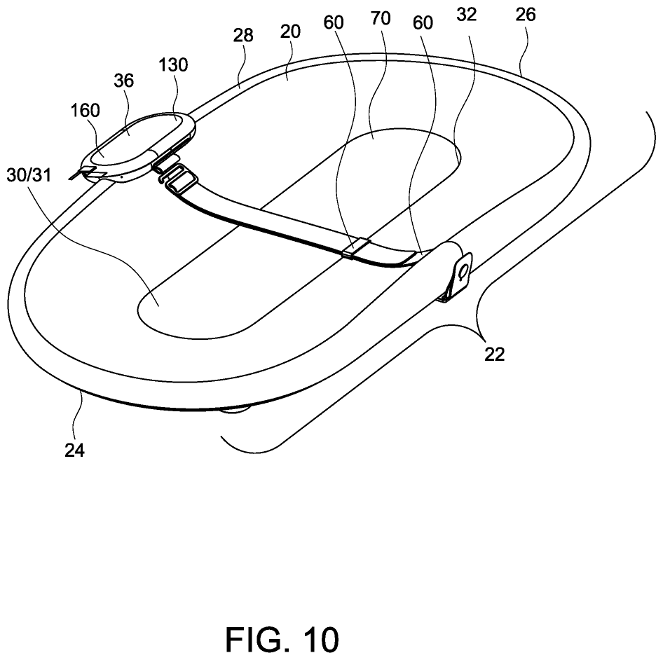

FIG. 10 shows an elevated front side view of an apparatus according to embodiments of the inventive subject matter;

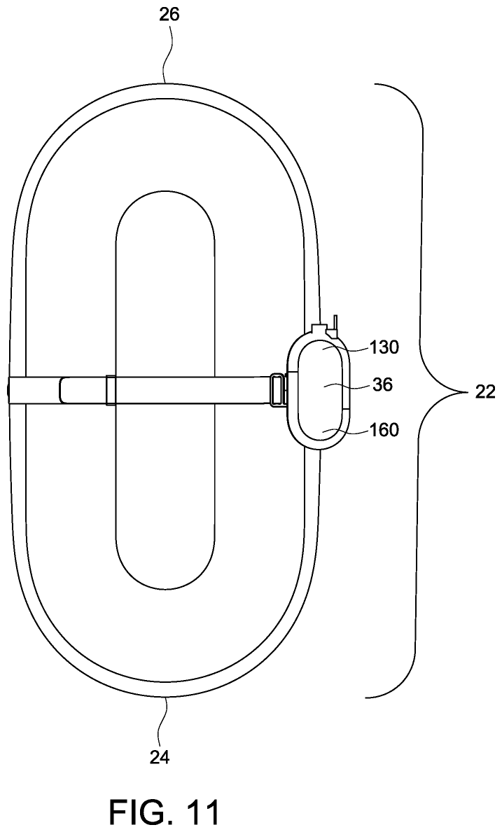

FIG. 11 shows a top view of an apparatus according to embodiments of the inventive subject matter;

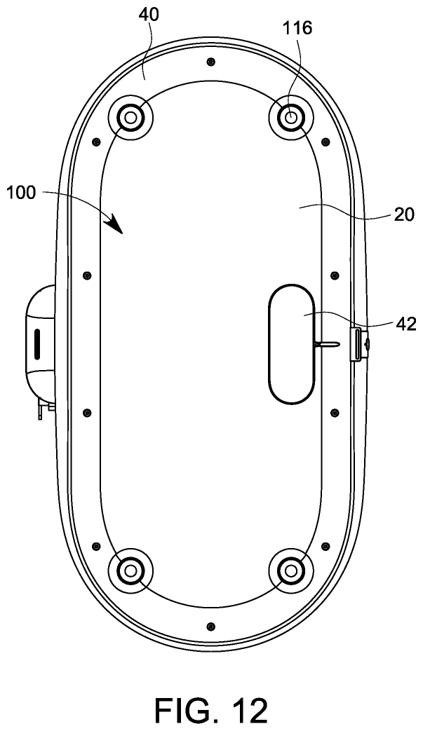

FIG. 12 shows a bottom view of an apparatus according to embodiments of the inventive subject matter;

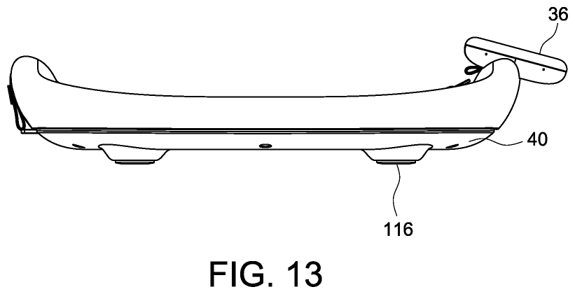

FIG. 13 shows a side view of an apparatus according to embodiments of the inventive subject matter;

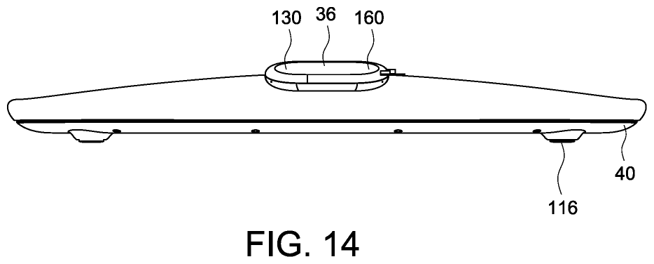

FIG. 14 shows another side view of an apparatus according to embodiments of the inventive subject matter;

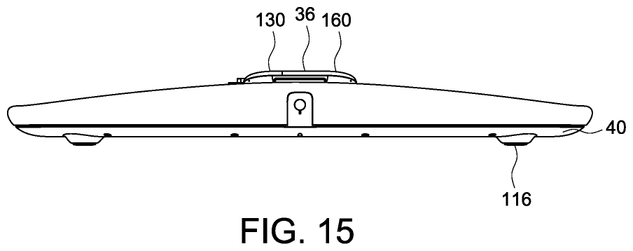

FIG. 15 shows a side view of an apparatus having a bracket according to embodiments of the inventive subject matter;

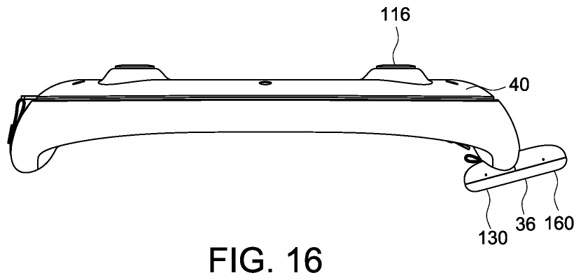

FIG. 16 shows a bottom side view of an apparatus according to embodiments of the inventive subject matter;

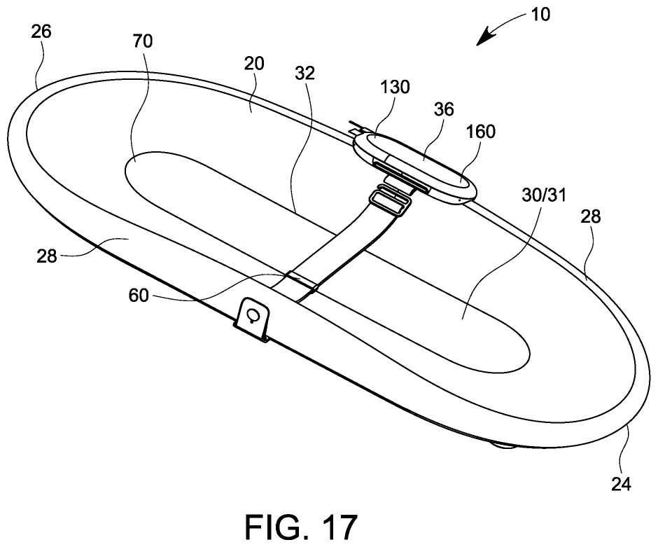

FIG. 17 shows an elevated side view of an apparatus according to embodiments of the inventive subject matter;

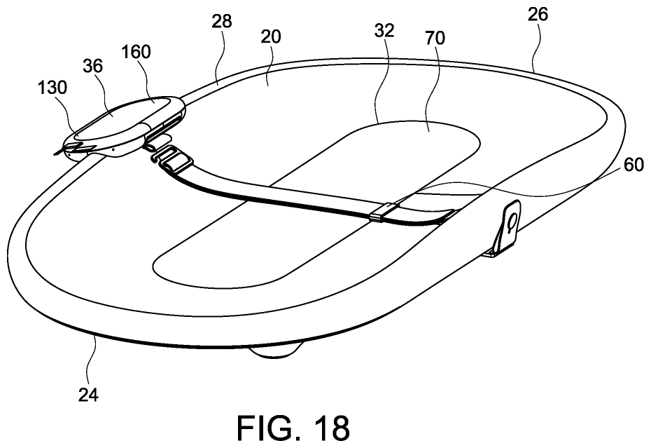

FIG. 18 shows an elevated rear view of an apparatus according to embodiments of the inventive subject matter;

FIG. 18 shows an under side view of an apparatus according to embodiments of the inventive subject matter;

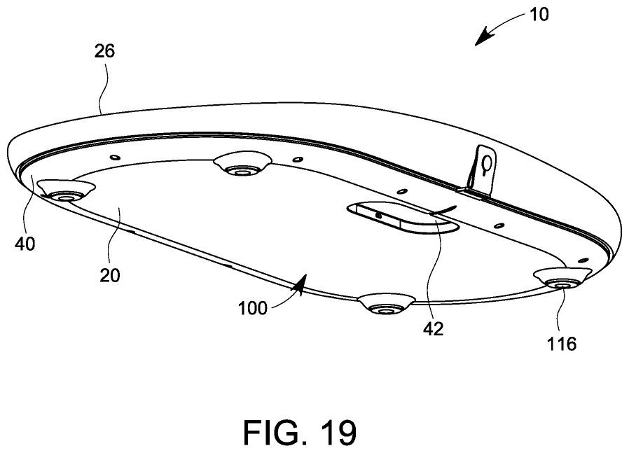

FIG. 19 shows another under side view of an apparatus according to embodiments of the inventive subject matter;

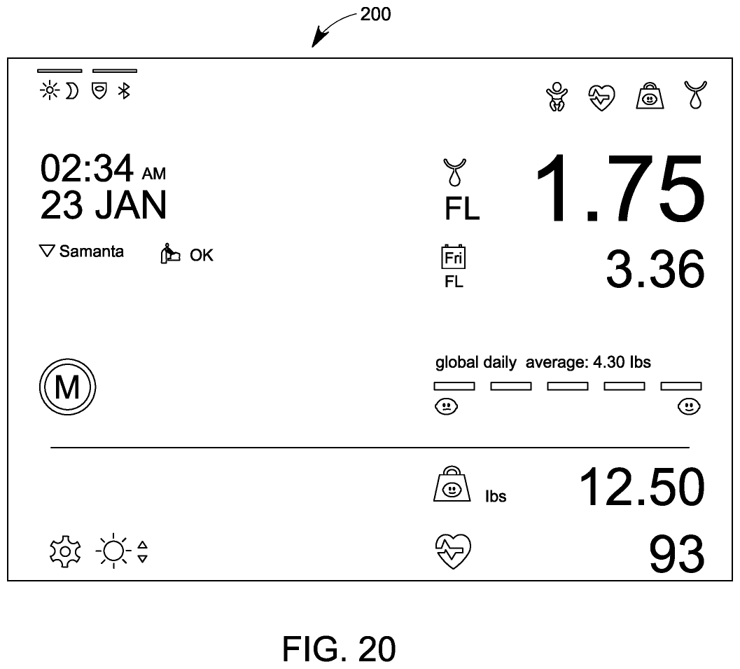

FIG. 20 shows an exemplary view of a display of an apparatus according to embodiments of the inventive subject matter;

### DETAILED DESCRIPTION OF THE EMBODIMENTS

In the following detailed description, reference is made to the accompanying drawings which form a part hereof, and in which is shown by way of illustration specific embodiments in which the disclosure may be practiced. These embodiments are described in sufficient detail to enable those skilled in the art to practice the disclosure, and it is to be understood that other embodiments may be utilized and that mechanical, procedural, and other changes may be made without departing from the spirit and scope of the disclosure. The following detailed description is, therefore, not to be taken in a limiting sense, and the scope of the disclosure is defined only by the appended claims, along with the full scope of equivalents to which such claims are entitled.

As used herein, the terminology such as vertical, horizontal, top, bottom, front, back, end, sides and the like are referenced according to the views, pieces and figures presented. It should be understood, however, that the terms are used only for purposes of example descriptions and are not intended to be used as limitations. Accordingly, orientation of an object or a combination of objects may change without departing from the scope of the claimed subject matter.

Reference throughout this specification to “one embodiment,” “an embodiment,” “one example,” or “an example” means that a particular feature, structure, or characteristic described in connection with the embodiment or example is included in at least one embodiment of the present disclosure. Thus, the appearance of the phrases “in one embodiment,” “in an embodiment,” “one example,” or “an example” in various places throughout this specification are not necessarily all referring to the same embodiment or example. Furthermore, the particular features, structures, databases, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

Embodiments in accordance with the present disclosure may be embodied as an apparatus, method, or computer program product. Accordingly, the present disclosure may take the form of an entirely hardware-comprised embodiment, an entirely software-comprised embodiment (including firmware, resident software, micro-code, etc.), or an embodiment combining software and hardware aspects that may all generally be referred to herein as a “circuit”, “module”, or “system”. Furthermore, embodiments of the present disclosure may include the form of a computer program product embodied in any tangible medium.

Any combination of one or more computer-usable or computer-readable media may be utilized. For example, a computer-readable medium may include one or more of a portable computer diskette, a hard disk, a random access memory (RAM) device, a read-only memory (ROM) device, an erasable programmable read-only memory (EPROM or Flash memory) device, a portable compact disc read-only memory (CDROM), an optical storage device, and a magnetic storage device. Computer program code for carrying out operations of the present disclosure may be written in any combination of one or more programming languages. Such code may be compiled from source code to computer-readable assembly language or machine code suitable for the device or computer on which the code will be executed.

Embodiments may also be implemented in cloud computing environments. In this description and the following claims, “cloud computing” may be defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction and then scaled accordingly. A cloud model can be composed of various characteristics (e.g., on-demand self-service, broad network access, resource pooling, rapid elasticity, and measured service), service models (e.g., Software as a Service (“Saas”), Platform as a Service (“PaaS”), and Infrastructure as a Service (“IaaS”)), and deployment models (e.g., private cloud, community cloud, public cloud, and hybrid cloud).

The flowchart and block diagrams in the attached figures illustrate the architecture, functionality, and operation of possible implementations of systems, methods, and computer implemented products according to various embodiments of the present disclosure. In this regard, each block in the flowchart or block diagrams may represent a module, segment, or portion of code, which comprises one or more executable instructions for implementing the specified logical function(s). It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions. These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flowchart and/or block diagram block or blocks.

All illustrations of the drawings are for the purpose of describing selected versions of the present disclosure and are not intended to limit the scope of the claimed subject matter.

The following descriptions are in reference to the figures. The present disclosure relates to an infant and child monitoring system, an intelligent changing pad system, a health tracking system, and methods of use. The present disclosure provides a novel and useful infant and child monitoring system. The present disclosure provides a new and useful intelligent changing pad system. The present disclosure provides a new and useful health tracking system. Furthermore, the present disclosure provides new and useful methods of use.

In the arrangement shown, as one embodiment, the present disclosure provides for an intelligent changing pad system. The intelligent changing pad system may be implemented when changing newborns, infants and/or children, as well as for other purposes. The present disclosure also provides for monitoring and tracking of infant and/or child health. In this way, the present disclosure provides for monitoring and tracking, especially important data, that a caregiver, medical provider and the like may need to assist in providing care for an infant and/or newborn and/or child.

Additionally, and in the arrangement shown, the present disclosure provides the state of the art with a safe intelligent changing pad system. Furthermore, in this way, the present disclosure provides the state of the art with a comfortable space for an infant and/or child. Furthermore, the present disclosure provides the state of the art with a comfortable space for an infant and/or child that is safe and comfortable.

Furthermore, the present disclosure provides the state of the art with a smart change pad that provides for tracking and/or monitoring infant and/or child growth. This is especially critical during the early period of human lives. Similarly, the present disclosure tracks and monitors this information for parents and/or caregivers and may also provide alerts and/or comparisons to normal data. Additionally, and similarly, the present disclosure provides a means for manual data entry so that various data can be tracked, analyzed, compared, and the like so that information can be processed and provided.

In one embodiment, the present disclosure provides a high accuracy scale, which is critical in the data tracking of newborns or infants. In this way, health and even small variations in the weight of an infant could be critical. Similarly, the high accuracy scale also helps track food intake and development of an infant and/or child, and the like.

Similarly, and in another example, the present disclosure also provides for graphical user interfaces, mobile phone applications, personal computer interfaces, tablet applications, and the like which provide for recording various information, storing this data, and providing this data to a user and/or healthcare provider, and/or subsequent and other caregivers. In addition to tracking measurements, weights, and the like, the present disclosure also provides event tracking and other similar data tracking, including but not limited to feeding occurrences, diaper change occurrences, as well as providing illustrations, videos and such to aid a user in the task at hand or in other learning tasks.

In this way, the present disclosure provides for overlay data, such as occurrences and weight, and provides further analysis and information to a user. Similarly, the system also provides knowledge and/or education to a user throughout a process if desired. Similarly, graphics and other helpful information such as imposed overlayment of a standard or a standard deviation in a graph and other display information is provided herein.

Furthermore, the present disclosure provides for additional components, features, and functionality including built-in interface and touchscreens providing for user input and other capabilities. Furthermore, the present disclosure provides for additional components, features and functionality directed toward visibility. These features include, but are not limited to, a night light and other viewing features that can aid a user in various environments, further easing stress, anxiety, and operation.

In the arrangement shown, as one example, the present disclosure also enhances the noise environment of a user and/or infant. These features include, but are not limited to, a white noise machine and components and other features which aid in creating a soothing environment.

Furthermore, the present disclosure provides for additional components, features and functionality directed toward temperature stimulus. These features include, but are not limited to, warming components, sensors, and features.

Furthermore, the present disclosure provides for additional components, features and functionality directed toward sanitary hygiene. In this way, some features include, but are not limited to a removable and washable cover and/or pad device that is easy to clean and improves upon overall health.

Furthermore, the present disclosure provides for additional components, features and functionality directed toward security and stability. In this way, some features include, but are not limited to securement features and the like.

Furthermore, the present disclosure provides the state of the art with support including but not limited to, recommendations, tips, insights, blogs, videos, local and national data, trends, patterns, video conferencing with physicians and others, among various other components, functionality, and features.

Although the disclosure may be explained in relation to examples of embodiments, it is to be understood that many other possible modifications and variations can be made without departing from the spirit and scope of the invention.

### System

With reference to the figures, an infant and child monitoring system, an intelligent changing pad system, a health tracking system, and methods of use 10 are presented (also referred to as “changing table system”, or “infant monitoring system”, or “child monitoring system”, or “intelligent changing table system”, or “intelligent changing pad system”, or “infant tracking system”, or “health tracking system”, or simply “system”).

Changing table system 10 is formed of any suitable size, shape and design and is configured to provide for health and data monitoring and tracking of an infant and/or child. In the arrangement shown, as one example, infant monitoring system 10, may comprise remote servers, databases, application servers, application databases, application programming interfaces, user databases, event databases, transmission rule databases, rules databases, data transmission components and/or features, mobile applications, and/or computers, and the like, that fulfill the functions disclosed herein.

Furthermore, interactive game system 10 may also include a user(s) 12, a plurality of infants 14 (or “child”, or “children”), a graphical user interface 16, a main body 20, a plurality of securement features 50, a changing pad 70, an bottom interaction system 100, a three-dimensional measurement system 130, a power supply 140, a computer system 150, a plurality of sensors 160, a warmer system 170, a vibration system 180, a display system 200, a sound system 210, a wireless communication system 220, and may also include a diaper dispenser system 230, a diaper disposal system 240, a wipe dispenser system, and may also include applications, application servers, application programming interfaces, a computer system, and a plurality of databases, among other features, components, and functionality.

FIGS. 1-20 show exemplary views of an apparatus 10 with various components indicated in numbers and FIG. 20 includes a display system 200 for displaying various information. In many embodiments, the display system 200 can display information related to a Bluetooth connection status, a protecting shield on or off status, a day or night mode indicator, a time and date display, a child or infant name display, a user profile, an child or infant profile, a confirmation that the measurement of the child or infant was successful, a display of measurements over time, for example displaying a fixed number of past measurements such as 10, 60, 120 or any suitable number or measurements over a fixed amount of time such as 10, 21, or 60 days or any suitable amount of time. In many embodiments, the display system 200 may also include global settings, administrator settings, update settings, screen brightness settings, night light settings, vibration settings, music settings, diaper changer tracking settings, pulse, weight display, units, sum of any suitable measurements over any suitable time period, a global data comparison such as weight and pulse display comparisons.

In the arrangement shown, as one example, intelligent changing table system 10 is an intelligent changing pad for use when changing newborns and infants. In the arrangement shown, as one example, system 10 provides a means for parents and other caregivers to monitor and track a newborn (or infants) basic health information and provide a safe and comfortable space for the infant and/or child during diaper changing and other health and/or maintenance items in caring for an infant and/or child.

In this way, and in the arrangement shown, as one example, a parent and/or caregiver can monitor and track infant and/or child growth, and the like, during various periods of a child's life, especially early periods of growth, which may be crucial to the health of an infant and/or child. Furthermore, in the arrangement shown, as one example (and as will be discussed further), system 10 provides for recording and data monitoring of various occurrences related to the health of an infant and/or child including, but not limited to feedings, diaper changes, growth rates, food consumption rates, and the like.

Furthermore, in the arrangement shown, as one example, the system 10 provides for tips and insights, and recommendations for new parents and/or caregivers to help aid in the success of caring. This includes, but is not limited to, providing articles, access to data, access to averages, access to national data, trends, patterns, and the ability to take notes related to this information and more.

### User:

In the arrangement shown, as one example, system 10 may include a user 12 and/or a plurality of users 12 (also known as “parent”, or “plurality of parents”, or “guardian”, or “care giver”). In the arrangement shown, a user may be an individual person, a group of people, and/or an entity such as a business, an organization or the like providing care to a newborn and/or an infant and/or a child.

In this way, and as one example, a user 12 and/or users 12 may be an individual.

In this way, an individual can engage with system 10 by caring for the infant and/or tracking information associated with the infant, via the system, benefiting from various activities and interactions with the system 10 which may enhance the wellbeing of the caregiver as well as enhance the wellbeing of the infant and/or child.

### Infant:

This term may be used to describe a type of person which data is collected on and/or interacts directly with system 10. In the arrangement shown, as one example, system 10 may include an infant 14 (also known as “child”, or “children”, or “newborn”). In the arrangement shown, a user will place an infant 14 on the changing pad system 10. In this way, a user can measure the infant 14, weigh the infant 14, change and/or maintain hygiene of the infant 14, and the like. In this way, an infant 14 may be a part of the system as the system is designed for interaction with an infant 14.

In this way, and as one example, a user 14 can engage with system 10 by caring for the infant and/or tracking information associated with the infant, via the system, benefiting from various activities and interactions with the system 10 which may enhance the wellbeing of the caregiver as well as enhance the wellbeing of the infant and/or child.

### Graphical User Interface:

In the arrangement shown, as one example, system 10 includes a graphical user interface 16. Graphical user interface 16 (also referred to as “interface system”, “display interface system”) is formed of any suitable size, shape, and design and is configured to present the user with an easy to use display system and easy to use system for monitoring and tracking data associated with infant care and health.

Additionally, graphical user interface 16 is configured to work over networks to share and enhance data, generate reports related to statistics tracked and/or health data which provide results and/or suggestions and the like. Said another way, graphical user interface 16 is configured as the primary interaction which allows the user to engage with system 10 in an electronic format as well as access the information remotely, and the like.

Additionally, graphical user interface 16 is configured to provide a plurality of users with access to these services, and alerts, and share health information and other information with physicians and others, in an easy to use way.

### Main Body:

In the arrangement shown, as one example, system 10 includes a main body 20 (also referred to as “changing table”, or “support assembly”). Main body 20 is formed of any suitable size, shape, and design and is configured as the primary structure of system 10.

In the arrangement shown, as one example, the main body 20 is generally flat and rectangular when viewed from the top. In the arrangement shown, as one example, the main body 20 extends a length 22 from a first end 24 (or bottom end) to a second end 26 (or top end), between opposing sides 28. The opposing sides 28 run in approximate parallel spaced relation and appear approximately rectangular in shape when viewed from the sides.

In the arrangement shown, as one example, the main body 20 also includes a top 30 (also referred to as “working surface”, or “bed”, or “table”, or “top surface”, or simply “surface”). Working surface 30 is formed of any suitable size, shape, and design and is configured as the primary working surface for which a changing pad may be integrated or laid upon. The top 30 is the main surface which an infant may be directly laying and/or sitting on. In the arrangement shown, as one example, the top surface 30 is generally rectangular when viewed from above. The surface 30 is generally concave and/or may include a concave surface 31. In this way, the surface 30 is designed to keep an infant from rolling out. This subtle feature, in this way, aids in both comfort and safety.

In the arrangement shown, as one example, the main body 20 also includes a plurality of guide features 32 (also referred to as “guides”, or “guards”, or “guard rails”). Guides 32 are formed of any suitable size, shape, and design and are configured for safety and comfort reasons as well as functional reasons. In the arrangement shown, as one example, the guides 32 are configured to keep the infant within the top surface 30 of the main body 20. In this way, the guides 32 are formed of vertically extending features which extend vertically from each of the opposing sides. Additionally, and in the arrangement shown, as one example, the guides 32 are integrated with a plurality of functional features, including but not limited to, a ruler and/or measurement system 33 and a lighting system 34, among other features, components and functionality. Guides 32 may also include strap and/or securement features and the like, among other integrations.

In the arrangement shown, as one example, the main body also includes a display surface 36. Display surface 36 is formed of any suitable size, shape, and design and is configured as the primary interaction surface of a user when viewing and/or tracking data and/or receiving alerts. In the arrangement shown, as one example, display surface 36 is a small vertically extending surface which is generally flat across the top and extends from the top end 26 (or second end 26) of the main body 20. In the arrangement shown, as one example, the display surface 36 includes a touchscreen and/or touch display which conveys information to a user and provides a means for a user to record and/or save information. Display surface 36, in the arrangement shown, as one example, also includes other flat surfaces, which aid in setting other items such as bottles, drinks, and the like.

In the arrangement shown, as one example, the main body 20 includes these and other features and also comprises an exterior surface 40, a hollow interior 42 and additional plugs as might be needed for power and other connection purposes such as phone charging, accessory plug in to provide power, and/or connection for transferring data and the like. Additionally, in the arrangement shown, as one example, the hollow interior 42 of the main body 20 is configured to house and hold a plurality of other components and systems which are included herein.

Furthermore, in the arrangement shown, as one example, the exterior surface 40 is formed of a polymer. However, other materials for the exterior surface 40 and other components are hereby contemplated for use. This may be a metal, an alloy, an advanced polymer, a carbon fiber, a fiber, a polyvinyl chloride, a wood, a combination thereof, or other suitable materials, which might include antimicrobial agents, antibacterial agents, some of which might be integrated and/or applied as a surface coating, and the like.

### Strap Assembly:

In the arrangement shown, as one example, system 10 includes a strap assembly 50 (also referred to as “securement assembly”, or “securement features”, or simply “straps”). Securement assembly 20 is formed of any suitable size, shape, and design and is configured to provide safety for an infant and user through securement of the infant and/or other devices on or around the system 10. In other words, the straps are configured to be used to safely and comfortably secure an infant to the top surface 30 of the main body 20 so the infant can not roll off the table if a caregiver turns away and the like.

In the arrangement shown, as one example, the strap assembly 50 extends a length from a first end 52 to a second end 56 and includes attachment features at each end 54/58. Additionally, the strap assembly may include a cushion or strap guard 60 running a portion of the strap assembly length or the duration of the strap assembly length. The cushion or strap guard is designed for comfort and ease of use. In the arrangement shown, as one example, the strap assembly may also include a retraction system 62 and/or winding system. In this way, the retraction system can safely stow the strap and/or plurality of straps when not in use. Additionally the retraction feature 62 can safely tighten and/or hold secure the strap and/or plurality of straps when in use.

### Changing Pad:

In the arrangement shown, as one example, system 10 includes a changing pad 70 (also referred to as “changing surface”, or “changing table”). Changing pad 70 is formed of any suitable size, shape, and design and is configured as a softened, more comfortable, surface for an infant to rest during the changing process or other processes. Changing pad 70 is also configured as an easy to clean surface which is also safe for an infant due to hygiene improvements. In the arrangement shown, as one example, the changing pad 70 is a separate component from the top surface 30 and designed to uniquely and safely fit and secure to the top surface 30 of the main body 20. However, the changing pad 70 may also be an integral part of the top surface 30 of the main body 20.

In the arrangement shown, as one example, the changing pad 70 is generally rectangular in shape when viewed from the top. In the arrangement shown, as one example, the changing pad 70 extends a length 72 from a first end 74 (or bottom end) to a second end 76 (or top end) and includes opposing sides 78 which extend in approximate parallel spaced relation to one another. In the arrangement shown, as one example, the changing pad 70 includes a top surface 80 (or working surface) and a bottom surface 82. In the arrangement shown, as one example, the changing pad may also include a bottom lip 84 so as to slightly overlap and fit snugly with the top surface 30 of the main body 20.

In the arrangement shown, as one example, the changing pad 70 is formed of a slightly cushioned material for comfort for the infant. The cushioned material may be wrapped in a cover or may be a standalone material. In the arrangement shown, as one example, the cover and/or the pad itself may include antimicrobial and/or antibacterial properties. Furthermore, the pad is designed and configured to be easily cleaned and wipeable. Furthermore, and in the arrangement shown, as one example, the pad 70 is removable and may include a plurality of attachment features 90 so the pad 70 can be repeatedly attached and/or removed from the main body 20 for cleaning and/or other purposes.

In the arrangement shown, as one example, changing pad 70 may also include a warming feature so the pad can be warmed for comfort. Additionally, the pad may include a plurality of wires 92, plugs 94, and the like as are contemplated for use for this functionality and others as further described herein.

### Base:

In the arrangement shown, as one example, system 10 includes a base 100 (also referred to as “base assembly”, or “base portion”). Base assembly 100 is formed of any suitable size, shape, and design and is configured to support system 10 and various components, which include balancing supports, strap attachment features, power features, and the like. Additionally, base 100 is formed of a hollow interior which may integrate with the hollow interior of the top such that the two parts or single part form a single hollow interior which houses and holds various components of system 10.

In the arrangement shown, as one example, the base is generally rectangular when viewed from the bottom and extends a length 102 from a first end 104 to a second end 106 between opposing sides 108 which extend in approximate parallel spaced relation. In the arrangement shown, as one example, the base assembly 100 also includes an exterior surface 110 and a hollow interior 112. In the arrangement shown, as one example, the base 100 also includes a plurality of adjustable support features 114. These adjustment features 114 can be adjusted for level and desired height. Additionally, the adjustment features may be automatically adjusted for safety and wobble reduction.

Similarly, and in the arrangement shown, as one example, the base 100 or system 10 may include a tilt feature 116 (also referred to as a “riser”). Tilt feature 116 is formed of any suitable size, shape, and design and is configured to automatically and or manually raise the upper portion of system 10. Said another way, the tilt feature 116 is configured to automatically and/or manually tilt system 10 such that an infant's head can be higher than its feet during operation. In this way, a number of benefits are gained, including reduction in spit up of an infant, and the like. Additionally, various attachment features 118 of the base and the like are shown and hereby contemplated for use.

### Three-Dimensional Measurement System:

In the arrangement shown, as one example, system 10 includes a three-dimensional measurement system 130 (also referred to as “measurement system”, or “size tracking system”). Three-dimensional measurement system 130 is formed of any suitable size, shape, and design and is configured to measure, monitor, and track the length and/or width of an infant. In this way, the three-dimensional measurement system 130 may be manual through a user using a ruler and/or the integrated ruler to determine length and/or width of the infant; subsequently recording that information on the display screen or application or computer system or the like.

Additionally, the system may include a plurality of sensors for automatically determining the dimensions of an infant and recording these dimensions in the tracking system. This action may be an automated action, or action completed by a predetermined and/or learned set of rules, when the table is in use or may be triggered by a user, and the like.

### Power Supply:

In the arrangement shown, as one example, system 10 includes a power supply 140 (also known as an “energy engine”, a “power engine”, an “energy supply”, a “battery”, a “plurality of batteries”, or simply an “engine”). Energy supply 140 is formed of any suitable size, shape, and design and is configured to provide energy to the system 10. Energy supply 140 is configured as a source of energy for system 10 and the various components, features, and functionality aspects of system 10 which may require power.

In the arrangement shown, as one example, power supply 140 is formed of corded power which supplies power to system 10 through a power plug into an outlet.

Additionally, and in the arrangement shown, as another example, power supply 140 is formed of a rechargeable battery. The rechargeable battery, or storage battery, is an electrical battery designed to be charged, discharged, and recharged as many times as needed, as opposed to a disposable or primary batter, which is disposed of after use. However, a primary battery or disposable battery is also hereby contemplated for use. The rechargeable battery, in the arrangement shown, is formed of one or more electrochemical cells. In the arrangement shown, as one example, power supply 140 is formed of a rechargeable battery. However, the power supply may also consist of a plurality of rechargeable batteries and/or a plurality of batteries.

Furthermore, in the arrangement shown, power supply 140 may also include all necessary components, whether wires, conductors, cases, holders, and the like which provide for necessary connections and stabilization for power supply 140 components.

### Computer System:

In the arrangement shown, as one example, system 10 includes a computer system 150 (also referred to as “processing system”, or “processing systems and subsystems”). Computer system 150 is further discussed herein.

### Sensors:

In the arrangement shown, as one example, system 10 includes a plurality of sensors 160 (also referred to as “sensor system”, or “sensors”). Sensory system 160 is formed of any suitable size, shape, and design and is configured to detect and/or analyze various environmental and system parameters.

In the arrangement shown, as one example, system 10 includes a sensor system 160. Sensor system 160 is formed of any suitable size, shape, and design and may include one or more sensors and/or one or more sensing technologies. In the arrangement shown, as one example, sensor system 160 is configured to detect various parameters including weight, temperature, size, safety warnings, heart rate, lighting, and the like.

In addition to the above mentioned and image sensors discussed herein, system 10 may also include other sensors, such as temperature sensors, moisture sensors, heat sensors, light sensors, motion sensors, and other sensors. In the arrangement shown, as one example, sensor system 160 is configured to facilitate sensing of the environment and various parameters and may also include LIDAR or other sensing equipment for tracking and/or monitoring. Both analog sensors and/or digital sensors are hereby contemplated for use. In one arrangement, another sensor and/or microsensor sends information to a processor for use with other electronics.

### Warmer System:

In the arrangement shown, as one example, system 10 includes a warming system 170. Warming system 170 is formed of any suitable size, shape, and design and is configured to warm. Said another way, the warming system 170 may be configured and designed to warm an infant while present on the table. Similarly the warming system 170 may be configured and designed to warm a bottle. Similarly, the warming system 170 may be configured to warm baby wipes, and the like.

### Vibration System:

In the arrangement shown, as one example, system 10 includes a vibration system 190. Vibration system 190 is formed of any suitable size, shape, and design and is configured to gently and safely provide vibration and/or movement to an infant. In this way, the vibration and/or movement can provide comfort for stress and anxiety reduction, sleeping aid, comfort aid, burping aid, and the like.

### Display System:

In the arrangement shown, as one example, system 10 includes a display system 200. Display system 200 is formed of any suitable size, shape, and design and is configured to provide information to a user. In the arrangement shown, as one example, display system 200 includes a touch screen built into the system 10. However, display system 200 may also include smartphone displays, tablet displays, computer displays, and other interaction and/or interactive devices for displaying and/or receiving information related to system 10.

### Sound System:

In the arrangement shown, as one example, system 10 includes a sound system 210. Sound system 210 is formed of any suitable size, shape, and design and is configured to provide sounds. Sound system is configured to provide white noise and/or other soothing sound effects such as waves, rain forest, cricket sounds and the like. In this way, sound system 210 is configured to comfort an infant and/or user. Additionally, sound system 210 can be used to play music and the like and may be bluetooth and/or wireless communication and/or wired communication enabled so that a user can play music and/or have phone calls and the like through sound system 210 (which may include speakers, microphones, and other wires and processing equipment).

### Wireless Communication System:

In the arrangement shown, as one example, system 10 includes a wireless communication system 220. Wireless communication system 220 is formed of any suitable size, shape, and design and is configured to provide communication means from system 10 to relay information related to information tracked and monitored. Wireless communication system 220 may include wifi and/or wireless communication through an internet and/or cellular connection. Additionally, wireless communication may include bluetooth, near field communication, and the like.

### Dispenser System:

In the arrangement shown, as one example, system 10 includes a dispenser system 230. Dispenser system 230 is formed of any suitable size, shape, and design and is configured to dispense. This may include dispensing diapers, wipes, and/or other items. Furthermore, dispenser system 230 may also include a warmer for the item being dispensed such as a wipe warmer system, and the like.

### Disposal System:

In the arrangement shown, as one example, system 10 includes a disposal system 240. Disposal system 240 is formed of any suitable size, shape, and design and is configured to provide a disposal and/or waste means. In the arrangement shown, as one example, the disposal system 240 is configured to accept waste, including but not limited to soiled diapers, and additionally can reduce smell and/or other escape once disposed.

### Application Programming Interface:

In the arrangement shown, as one example, system 10 may also include one or more application programming interfaces (“API”).

### Application Server:

In the arrangement shown, as one example, system 10 comprises remote servers, databases, and/or computers that fulfill the functions disclosed and described herein. In the embodiment depicted, system 10 comprises at least one application server. Application server comprises one or more computer systems adapted to transmit and receive data regarding selected datasets related to various users and/or datasets related to multiple users. Application server is adapted to a query database with unique identification codes to retrieve data information and/or parameters related to users, infants, items, data, health information, health data, textual responses, and more.

Application servers may transmit user data related to layers and rules with respect to a single user and/or multiple users or a single infant and/or multiple infants. Application server is also adapted to query a user database. This query includes receiving and sending user identification codes and user data and/or textual responses. Additionally, the application server may communicate with a mobile application, which is adapted to present the user information in a form conducive to being viewed on a mobile device and/or handheld device.

As one of ordinary skill in the art may understand, application server, project database, and other databases mentioned herein may be implemented in one or more servers. Furthermore, each may be on multiple servers to increase system efficiency, especially when handling large data gathering, data organizing, such as handling global positioning of a user, following extended rules for various functionality of system 10 and/or processing, updating user information, including various responses entered.

Additionally, multiple servers may have mirrored data to prevent data loss in case of disk failure and/or to decrease access and response times for database queries. In alternative embodiments, application server, and other database procedures may be carried out on computer-readable instructions and data stored on the customer's mobile computing device.

Additionally, system 10 may include a remote server, a computing system, includes an application programming interface (“API”) which includes tools and resources enabling a user to operate the embodiments herein, and a cloud computing system.

### Computing System:

In one arrangement, as is shown, smart devices and/or system 10 includes computing systems. Computing system is formed of any suitable size, shape, and design and configured to handle computing operations, as are necessary for the operation of the computing functionality of system 10. Computing system may be connected with an electronic network and/or database and/or server or cloud via communication means and includes a processor, a memory, a microcontroller, a printed circuit board, a microprocessor, a receiver/transceiver, among other components.

Computing devices may be formed of any computing device capable of displaying and manipulating data in the manners described herein. Computing devices may include for example a desktop computer, a laptop computer, a tablet, smart phone, or any other computing device or other interactive device.

Computing devices may be a single consolidated component, or alternatively, computing devices may be formed of a plurality of interconnected components that may be co-located or located at different geographic locations. Computing devices may be cloud based or it may be hardware based, or cloud capable. In addition, the connected components of computing devices, including processor, memory, software and interactive user display, may be co-located with computing devices or located at different geographic locations. That is, computing devices may be made of any form of a device or system that individually or collectively performs the computing operations of system 10.

Printed Circuit Board: In the arrangement shown, as one example, system 10 includes a printed circuit board (“PCB”). PCB is formed of any suitable size, shape and design and is configured to facilitate carrying and/or holding other components and/or parts necessary to carry out various computation and/or related functions of system 10. PCB, as one example, might be a surface mounted PCB or a through-hole PCB. PCB, as one example, is green and facilitates connecting the components and/or parts of system 10 by the use of traces and or vias. Traces are formed of any suitable size, shape and design and are configured as lines electrically connecting the components and/or parts of system 10. Vias are formed of any suitable size, shape and design and are configured as holes that connect layers of traces together. Generally, as in shown, traces and vias are soldered to connect the components and/or parts to the PCB.

Microprocessor: Microprocessor is any computing device that receives and processes information and outputs commands according to instructions stored in memory. Memory is any form of information storage such as flash memory, RAM memory, a hard drive, or any other form of memory. Memory may be included as a part of or operably connected to a microprocessor. A receiver/transceiver is connected to a microprocessor. A receiver is used if one way communication is utilized, whereas a transceiver is used if two-way communication is utilized (hereinafter “transceiver”).

Memory: In the arrangement shown, as on example, system 10 includes a memory. Memory may be formed of any suitable size, shape and design and is configured to facilitate selective storage and retrieval of data (including data) in association with computing devices, processor, software and interactive user display. Memory may be a single component, such as a single chip or drive or other memory device, or alternatively memory may be formed of a plurality of memory or storage components that are connected to one another that may be co-located or located at different geographic locations.

### Methods of Use:

As one example, an infant and child monitoring system, an intelligent changing pad system, a health tracking system, and methods of use are presented. The intelligent changing table system is further described as a method of use in the following examples of methods of use. These and other methods of use and processes are hereby contemplated for use.

Additional embodiments include EMR integration. These embodiments can additionally be used for integration into EMRs to securely transmit child vitals to pediatrician and health systems. These types of data can include a child's sleep schedule, feeding, diaper changes (including content of diapers) and weight data.

Additional embodiments include expanding the Child Vitals. In these embodiments, additional child vitals are collected. These include but are not limited to height, head circumference and development milestones such as crawling, walking, potty training, sleep training and talking.

In many embodiments, onboard, integrated or offboard in communication artificial intelligence agents, systems, devices or code (AI) can continuously monitor the child and care giver's inputs as well as provide feedback to the system and/or the user with optional alerts as desired to one or more parties.

In many embodiments, the child or infant's health vitals can be collected with one or more sensors. These embodiments can monitor one or more thermometers (such as integrated or non-integrated thermometers,) blood oximeters, heartbeat monitors, blood pressure monitors, and bilirubin monitors among others. Additional embodiments can use one or more sensors to monitor any readings known to those skilled in the art.

AI Signals: In many embodiments, the AI can be used to conduct sentiment analysis, language proficiency, any other types of analysis of data including language proficiency analysis and comprehension level analysis. For example, the AI used with many of the embodiments can analyze a child's crying to determine whether or not the crying is occurring due to illness, a need for food, or any other needs.

In many embodiments, clinical support models can be developed based on the data collected in order to determine and provide early signals to medical professionals of potential risks with a child or family. These can be used in a number of ways to promote health in general to the population.

Many embodiments offer parental support. For example, embodiments can support models which can speak different languages in order to improve the reach in support of basic child care services to underserved communities.

Many embodiments can use over the air updates similar to the updated used with the popular Tesla® branded cars. With these updates, new features, updated and capabilities can be uploaded to the embodiment's software directly via the cloud. This can lead to improved functionality as well as new functionality. The embodiments also can communicate with the manufacturer and/or distributors so that the embodiments can be monitored which can lead to improve usability of the embodiments.

Streaming Media: Many embodiments can also stream video, audio and text content directly to the embodiments, for example directly to the device or apparatus and/or the app used with the embodiments. For example, when a parent needs to know how to change a diaper or play music, the embodiment can allow the parent to view and/or hear content directly on their personal electronic device and/or the display or app used with the embodiment.

Personalized Media: Many of the embodiments can train the AI to speak in the voice of the parent. This allows the parent voice to read a book, sing a lullaby or speak to the audience without the parent being present. Many embodiments allow a parent or other user to choose any suitable voice such as a known cartoon character or another family member.

It will be appreciated by those skilled in the art that other various modifications could be made to the disclosed embodiments without parting from the spirit and scope of the inventive subject matter in the present disclosure including various programmable features. All such modifications and changes fall within the scope of the claimed subject matter and are intended to be covered thereby.

### REFERENCE NUMERALS

10—System
12—User
14—Infant/Child
16—Graphical User Interface
20—Main Body 22—Length
24—First End (bottom) 26—Second End (top) 28—Opposing Sides
30—Top (working surface)
31—Concave Surface
32—Guard Rails (of 30)
33—Ruler (measurement feature)
34—Lighting System (of 30)
36—Display Surface (of 30)
38—Display (of 36) 40—Exterior Surface 42—Hollow Interior 44—Additional plugs
50—Strap Assembly 52—First End
54—Attachment Features 56—Second End
58—Attachment Features 60—Cushion/Strap Guard 62—Retraction System
70—Changing Pad 72—Length
74—First End 76—Second End
78—Opposing Sides
80—Top (working surface) 82—Bottom
84—Bottom Lip
86—Main Body (curved)
88—Antimicrobial Properties 90—Attachment Features
92—Wires 94—Plugs
100—Base 102—Length 104—First End
106—Second End 108—Opposing Sides
110—Exterior Surface 112—Hollow Interior
114—Adjustable Support Features 116—Tilt Feature (riser)
118—Attachment Features
130—Three-dimensional measurement system 140—Power Supply
150—Computer System
160—Sensors
162—Temperature 164—Heart Rate 166—Weight
168—Size
170—Lighting System 180—Warmer System 190—Vibration System
200—Display System 202—Touchscreen
210—Sound System
220—Wireless Communication System 230—Diaper Dispenser System
240—Diaper Disposal System
250—Wipe System 252—Wipe Dispenser 254—Wipe Warmer
300—Application Programming Interface
400—Application Server 500—Server

What is claimed is:

1. An intelligent changing pad system for infants and children, comprising:
a main body with a changing surface;
a scale integrated within the changing surface for measuring weight;
a plurality of sensors configured to monitor health-related metrics of a human including temperature and hydration levels;
a graphical user interface for displaying the human's health metrics and alerts; and
a wireless communication system for transmitting the monitored data to a remote computing device.

2. The system of claim 1, wherein the graphical user interface is also configured to display growth tracking information of the human over time.

3. The system of claim 1, further comprising a removable cover for the changing surface.

4. The system of claim 1, wherein the plurality of sensors includes a moisture sensor for detecting wetness.

5. The system of claim 1, further comprising a vibration system integrated within the main body for soothing the human.

6. The system of claim 1, wherein the human is a non-human animal.

7. The system of claim 1, wherein the scale is capable of measuring weight to within a gram.

8. The system of claim 1, further including a temperature control system for adjusting the surface temperature of the changing pad.

9. The system of claim 1, further including a sound system for playing soothing sounds or white noise.

10. The system of claim 1, wherein the wireless communication system includes Bluetooth connectivity for communicating with one or more mobile devices.

11. The system of claim 1, further including a night light feature integrated into the main body for illumination.

12. The system of claim 1, wherein the health-related metrics further include heartbeat and breathing rates of the human.

13. The system of claim 1, further including a securement system to safely hold the human in place on the changing surface.

14. The system of claim 1, wherein the graphical user interface is configurable for multiple users to track health metrics for more than one child.

15. The system of claim 1, further including a power supply system comprising a rechargeable battery for portable use.

16. The system of claim 1, wherein the remote computing device includes a mobile application for real-time monitoring and alerts.

17. The system of claim 1, further comprising a disposal system for used diapers integrated into or adjacent to the main body.

18. A method for monitoring and managing the health and hygiene of an infant or child using an intelligent changing pad system, comprising the steps of:
placing the infant or child on a changing pad integrated with sensors;
measuring health-related metrics via said sensors, including at least weight and temperature;
transmitting the measured metrics to a data processing unit;
analyzing the metrics in comparison to predefined health benchmarks;
generating health insights based on the analysis;
displaying the health insights on a graphical user interface; and
communicating alerts or recommendations to caregivers via a connected mobile application.

19. The method of claim 18, further comprising the step of customizing the health insights based on the infant's or child's age and development stage.

20. An infant or child care apparatus comprising:
a main body with a supportive surface configured to accommodate an infant or child for care activities;
an integrated high-accuracy scale for determining and recording weight;
a plurality of sensors for monitoring health-related metrics including temperature, hydration levels, and growth patterns;
a graphical user interface for displaying one or more of: monitored metrics, health insights, and care recommendations;
a wireless communication module for transmitting data to a remote computing device;
a changeable and washable surface covering for maintaining hygiene; and
a temperature control system for adjusting the surface temperature, wherein the apparatus is configured to provide a monitoring, tracking, and care management solution for infants and children.
